(19) United States
(12) Patent Application Publication
(10) Pub. No.: US 2025/0032009 A1
(43) Pub. Date: Jan. 30, 2025

(54) HIGH-PRECISION SMART RING

(71) Applicant: JIANHUI WU, Jingdezhen (CN)

(72) Inventor: JIANHUI WU, Jingdezhen (CN)

(21) Appl. No.: 18/645,827

(22) Filed: Apr. 25, 2024

(30) Foreign Application Priority Data
Jul. 28, 2023 (CN) 202322009369.6

(51) Int. Cl. A61B 5/145; A61B 5/00

(52) U.S. Cl. CPC A61B 5/14542; A61B 5/6826; A61B 2562/0271

(57) ABSTRACT

Disclosed is a high-precision smart ring. Blood oxygen emitters are disposed on both sides of a blood oxygen receiver. The axial distance d between the blood oxygen emitter and the blood oxygen receiver at an inner-side wall surface of a ring holder is kept between 3.8 mm and 7 mm. In addition, a light shield is covered on the periphery of the blood oxygen receiver for preventing infrared light generated by a light source of the blood oxygen emitter from interfering with the receiving accuracy of the blood oxygen receiver, thereby greatly improving the accuracy of the heart rate and blood oxygen sensor. Then, the electrical devices on a flexible circuit board are arranged circumferentially around the ring holder in the sequence of the blood oxygen emitter, the blood oxygen receiver, blood oxygen emitter, a main control chip, an antenna, a storage battery, a charging interface and a temperature sensor.

### CROSS-REFERENCE TO RELATED APPLICATIONS

The application claims priority to Chinese patent application No. 202322009369.6, filed on Jul. 28, 2023, the entire contents of which are incorporated herein by reference.

### TECHNICAL FIELD

The present disclosure relates to the technical field of smart devices, specifically relates to a high-precision smart ring.

### BACKGROUND

With the development of Internet technology and intelligent products, a variety of intelligent wearing products have emerged on the market. Various intelligent wearing products are used to achieve various detection and monitoring functions, and combined with the Internet to achieve intelligentization. Different from traditional rings, smart rings have different electronic functions and can be flexibly designed according to requirements. The common smart ring integrates a flexible circuit board and various sensors inside the ring, and can monitor various physical sign data of the human body via the sensors. Among them, the most commonly used sensor is currently a heart rate and blood oxygen sensor. The heart rate and blood oxygen sensor includes a blood oxygen emitter and a blood oxygen receiver. The blood oxygen emitter emits infrared light to the finger of the human body. After the infrared light passes through the blood vessel, a part of the light will be absorbed, and another part will pass through the blood vessel and irradiate onto a photosensitive resistor of the blood oxygen receiver. Due to different oxygen contents, the absorption degree of the light by the blood is also different. Thus, the oxygen saturation can be determined by detecting the change in light intensity received by the photoresistor. During the measurement, the blood oxygen emitter will continuously emit infrared light, and also record the light intensity received by the photoresistor, so as to finally give the value of oxygen saturation by making calculation through a certain algorithm.

At present, the monitoring data of smart rings on the market are often not accurate enough, which is caused by the unscientific sensor setting. Therefore, it is particularly important to improve the accuracy of smart ring monitoring by combining characteristics of human body and the principle characteristics of sensors.

### SUMMARY

The purpose of the present disclosure is to provide a high-precision smart ring, which can greatly improve the monitoring accuracy of a heart rate and blood oxygen sensor by reasonably arranging the orientation relationship between a blood oxygen emitter and a blood oxygen receiver in the heart rate and blood oxygen sensor of the smart ring by a large number of practices combined with scientific theoretical research, and solve the problems raised in the above-mentioned background art.

In order to achieve the above object, the present disclosure provides the following technical solutions. A high-precision smart ring comprises a ring holder, wherein an accommodating cavity is formed on the outer circumferential side of the ring holder; a flexible circuit board is laid in the accommodating cavity; a blood oxygen emitter and a blood oxygen receiver are electrically connected to and disposed on the flexible circuit board; a main control chip, an antenna, a storage battery, a charging interface and a temperature sensor are disposed in the accommodating cavity; and the main control chip, the antenna, the storage battery, the charging interface and the temperature sensor are all electrically connected to the flexible circuit board.

Preferably, an blood oxygen window is disposed on an inner-side wall surface of the ring holder; the blood oxygen emitter and the blood oxygen receiver are disposed at the blood oxygen window and towards the circle center of the ring holder; and the axial distance d between the blood oxygen emitter and the blood oxygen receiver at the inner-side wall surface of the ring holder satisfies 3.8 mm≤ d≤7 mm.

Preferably, the blood oxygen emitters are provided in two; the blood oxygen receivers are provided in one; the two blood oxygen emitters are symmetrically disposed on both sides of the blood oxygen receiver.

Preferably, the blood oxygen receiver is circumferentially covered with a light shield.

Preferably, the light shield is formed and coated by a light shielding coating.

Preferably, the blood oxygen emitter, the blood oxygen receiver, the blood oxygen emitter, the main control chip, the antenna, the storage battery, the charging interface and the temperature sensor are arranged circumferentially around the ring holder in sequence.

Preferably, the ring holder is provided with a through hole at a position corresponding to the charging interface; and the charging interface is disposed in the through hole.

Preferably, the ring holder is a thermally conductive metal ring; and the temperature sensor is thermally conductively connected to the ring holder.

Preferably, the accommodating cavity is filled with a potting adhesive; and the potting adhesive is provided in a way of submerging the blood oxygen emitter, the blood oxygen receiver, the main control chip, the antenna, the storage battery, the charging interface, the temperature sensor and the flexible circuit board.

Preferably, a decorative panel is annularly disposed at a cavity opening of the accommodating cavity; the potting adhesive is provided in a way of submerging the outer surface of the decorative panel; and the potting adhesive is a transparent potting adhesive.

The present disclosure has the following beneficial effects compared to prior art.

In the present disclosure, blood oxygen emitters are disposed on both sides of a blood oxygen receiver. The axial distance d between the blood oxygen emitter and the blood oxygen receiver at an inner-side wall surface of a ring holder is kept between 3.8 mm and 7 mm. In addition, a light shield is covered on the periphery of the blood oxygen receiver for preventing infrared light generated by a light source of the blood oxygen emitter from interfering with the receiving accuracy of the blood oxygen receiver, thereby greatly improving the accuracy of the heart rate and blood oxygen sensor. Then, the electrical devices on a flexible circuit board are arranged circumferentially around the ring holder in the sequence of the blood oxygen emitter, the blood oxygen receiver, blood oxygen emitter, a main control chip, an antenna, a storage battery, a charging interface and a temperature sensor, so that the radiated interference of each electrical device can be avoided reasonably in the narrow and limited ring space, which is beneficial to improve the stability of each sensor and further improve the accuracy of smart ring monitoring.

### BRIEF DESCRIPTION OF DRAWINGS

FIG. 1 is a structurally exploded view of the present disclosure;

FIG. 2 is a structurally schematic view of the present disclosure; and

FIG. 3 is a layout schematic view of internal electrical parts of the present disclosure.

In the drawings, 1, ring holder; 11, blood oxygen window; 2, flexible circuit board; 21, blood oxygen emitter; 22, blood oxygen receiver; 221, light shield; 23, main control chip; 24, antenna; 25, storage battery; 26, charging interface; 27, temperature sensor; 3, potting adhesive; 4, decorative panel.

### DESCRIPTION OF EMBODIMENTS

The technical solutions in the embodiments of the present disclosure will be described clearly and completely in conjunction with the accompanying drawings in the embodiments of the present disclosure. Obviously, the described embodiments are only part of the embodiments of the present disclosure, rather than all of the embodiments. Based on the embodiments in the present disclosure, all other embodiments obtained by a person skilled in the art without involving any inventive effort are within the scope of protection of the present disclosure.

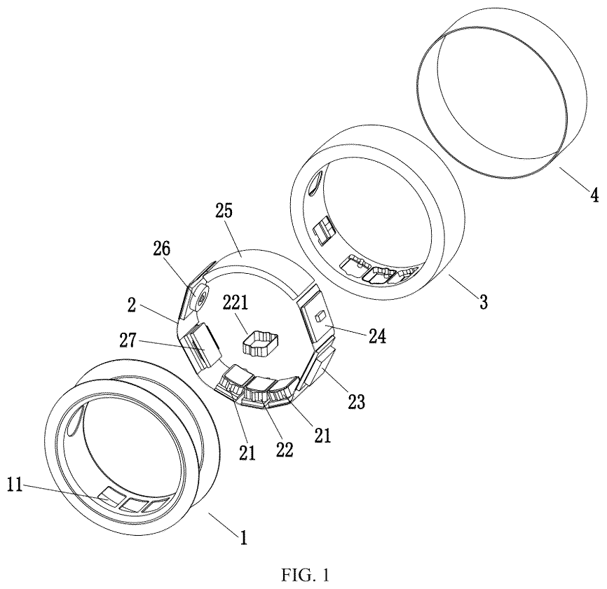

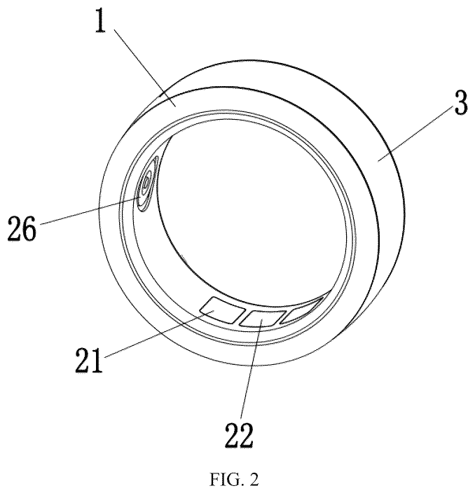

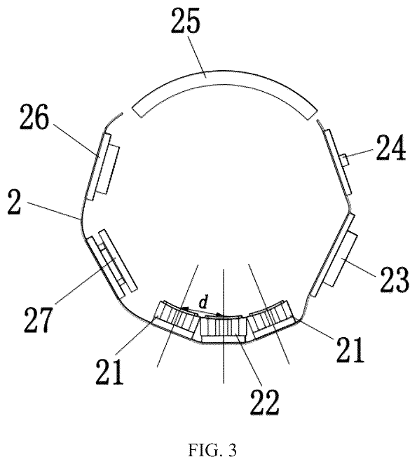

With reference to FIGS. 1-3, a high-precision smart ring includes a ring holder 1. An accommodating cavity is formed on the outer circumferential side of the ring holder 1. A flexible circuit board 2 is laid in the accommodating cavity. A blood oxygen emitter 21 and a blood oxygen receiver 22 are electrically connected to and disposed on the flexible circuit board 2. A blood oxygen window 11 is disposed on an inner-side wall surface of the ring holder 1. The blood oxygen emitter 21 and the blood oxygen receiver 22 are disposed at the blood oxygen window 11 and towards the circle center of the ring holder 1. The axial distance d between the blood oxygen emitter 21 and the blood oxygen receiver 22 at the inner-side wall surface of the ring holder 1 satisfies 3.8 mm≤d≤7 mm.

The blood oxygen emitters 21 are provided in two. The blood oxygen receivers 22 are provided in one. The two blood oxygen emitters 21 are symmetrically disposed on both sides of the blood oxygen receiver 22. The blood oxygen receiver 22 is circumferentially covered with a light shield 221. The light shield 221 is formed and coated by a light-shielding coating or a light-proof shell material.

A main control chip 23, an antenna 24, a storage battery 25, a charging interface 26 and a temperature sensor 27 are disposed in the accommodating cavity. The main control chip 23, the antenna 24, the storage battery 25, the charging interface 26 and the temperature sensor 27 are all electrically connected to the flexible circuit board 2, and are arranged circumferentially around the ring holder 1 according to the sequence of blood oxygen emitter 21, the blood oxygen receiver 22, the blood oxygen emitter 21, the main control chip 23, the antenna 24, the storage battery 25, the charging interface 26 and the temperature sensor 27. The ring holder 1 is provided with a through hole at a position corresponding to the charging interface 26. The charging interface 26 is disposed in the through hole. The ring holder 1 is a thermally conductive metal ring. The temperature sensor 27 is thermally conductively connected to the ring holder 1.

During the assembling and processing, each electrical device is firstly assembled on the flexible circuit board 2, and then the flexible circuit board 2 is fixed at an outer wall surface of the ring holder 1. Later, a potting adhesive 3 is filled into the accommodating cavity, where the potting adhesive 3 is provided in a way of submerging the blood oxygen emitter 21, the blood oxygen receiver 22, the main control chip 23, the antenna 24, the storage battery 25, the charging interface 26, the temperature sensor 27 and the flexible circuit board 2. This is a first glue filling process. After the potting adhesive of the first potting adhesive is solidified, the decorative panel 4 is wound around the periphery of the potting adhesive 3, and then a second glue filling process is performed. The potting adhesive 3 is provided in a way of submerging the outer surface of the decorative panel 3, and the potting adhesive 3 is a transparent potting adhesive. After the second potting adhesive is solidified, a sander is used for shaping to finally form a finished smart ring with a smooth and transparent outer surface and capable of making the decorative panel 3 perspective.

In summary, in the present disclosure, blood oxygen emitters 21 are disposed on both sides of a blood oxygen receiver 22. The axial distance d between the blood oxygen emitter 21 and the blood oxygen receiver 22 at an inner-side wall surface of a ring holder is kept between 3.8 mm and 7 mm. In addition, a light shield 221 is covered on the periphery of the blood oxygen receiver 22 for preventing infrared light generated by a light source of the blood oxygen emitter 21 from interfering with the receiving accuracy of the blood oxygen receiver 22, thereby greatly improving the accuracy of the heart rate and blood oxygen sensor. Then, the electrical devices on a flexible circuit board 2 are arranged circumferentially around the ring holder 1 in the sequence of the blood oxygen emitter 21, the blood oxygen receiver 22, blood oxygen emitter 21, the main control chip 23, the antenna 24, the storage battery 25, the charging interface 26 and the temperature sensor 27, so that the radiated interference of each electrical device can be avoided reasonably in the narrow and limited ring space, which is beneficial to improve the stability of each sensor and further improve the accuracy of smart ring monitoring.

It should be noted that relational terms such as first and second, and the like, may be used herein to distinguish one entity or action from another entity or action without necessarily requiring or implying any such actual relationship or order between such entities or actions. Furthermore, the terms “comprises”, “comprising”, or any other variation thereof, are intended to cover a non-exclusive inclusion, such that a process, method, article, or apparatus that includes a list of elements does not include only those elements but may include other elements not expressly listed or inherent to such process, method, article, or apparatus.

While embodiments of the present disclosure have been shown and described, it will be understood by those skilled in the art that various changes, modifications, substitutions and alterations may be made herein without departing from the principles and spirit of the present disclosure, the scope of which is defined by the appended claims and their equivalents.

What is claimed is:

1. A high-precision smart ring, comprising a ring holder (1), wherein an accommodating cavity is formed on the outer circumferential side of the ring holder (1); a flexible circuit board (2) is laid in the accommodating cavity; a blood oxygen emitter (21) and a blood oxygen receiver (22) are electrically connected to and disposed on the flexible circuit board (2); a main control chip (23), an antenna (24), a storage battery (25), a charging interface (26) and a temperature sensor (27) are disposed in the accommodating cavity; and the main control chip (23), the antenna (24), the storage battery (25), the charging interface (26) and the temperature sensor (27) are all electrically connected to the flexible circuit board (2).

2. The high-precision smart ring according to claim 1, wherein an blood oxygen window (11) is disposed on an inner-side wall surface of the ring holder (1); the blood oxygen emitter (21) and the blood oxygen receiver (22) are disposed at the blood oxygen window (11) and towards the circle center of the ring holder (1); and the axial distance d between the blood oxygen emitter (21) and the blood oxygen receiver (22) at the inner-side wall surface of the ring holder (1) satisfies 3.8 mm≤d≤7 mm.

3. The high-precision smart ring according to claim 1, wherein the blood oxygen emitters (21) are provided in two; the blood oxygen receivers (22) are provided in one; the two blood oxygen emitters (21) are symmetrically disposed on both sides of the blood oxygen receiver (22).

4. The high-precision smart ring according to claim 1, wherein the blood oxygen receiver (22) is circumferentially covered with a light shield (221).

5. The high-precision smart ring according to claim 4, wherein the light shield (221) is formed and coated by a light shielding coating.

6. The high-precision smart ring according to claim 1, wherein the blood oxygen emitter (21), the blood oxygen receiver (22), the blood oxygen emitter (21), the main control chip (23), the antenna (24), the storage battery (25), the charging interface (26) and the temperature sensor (27) are arranged circumferentially around the ring holder (1) in sequence.

7. The high-precision smart ring according to claim 1, wherein the ring holder (1) is provided with a through hole at a position corresponding to the charging interface (26); and the charging interface (26) is disposed in the through hole.

8. The high-precision smart ring according to claim 1, wherein the ring holder (1) is a thermally conductive metal ring; and the temperature sensor (27) is thermally conductively connected to the ring holder (1).

9. The high-precision smart ring according to claim 1, wherein the accommodating cavity is filled with a potting adhesive (3); and the potting adhesive (3) is provided in a way of submerging the blood oxygen emitter (21), the blood oxygen receiver (22), the main control chip (23), the antenna (24), the storage battery (25), the charging interface (26), the temperature sensor (27) and the flexible circuit board (2).

10. The high-precision smart ring according to claim 9, wherein a decorative panel (4) is annularly disposed at a cavity opening of the accommodating cavity; the potting adhesive (3) is provided in a way of submerging the outer surface of the decorative panel (3); and the potting adhesive (3) is a transparent potting adhesive.
